(19) United States
(12) Patent Application Publication
(10) Pub. No.: US 2014/0150155 A1
(43) Pub. Date: Jun. 5, 2014

(54) EYE COVER

(71) Applicant: Cecil Sessions, Miami Beach, FL (US)

(72) Inventor: Cecil Sessions, Miami Beach, FL (US)

(21) Appl. No.: 13/693,920

(22) Filed: Dec. 4, 2012

(51) Int. Cl. A61F 9/04

(52) U.S. Cl. CPC A61F 9/045
USPC 2/15

(57) ABSTRACT

An eye cover particularly adapted to hold a slice of fruit or other treatment against the eyelid and face during a health and beauty treatment. The device comprises an adjustable headband that secures a pair eyepieces over the eye sockets. The eyepieces are connected over the center of the face at a bridge. Variations of the device include a rim to hold the fruit on and a rim-less design. Optional graphics are provided to customize the look of the device.

### BACKGROUND OF THE INVENTION

1. Field of the Invention

The present invention relates to eye covering, and more particularly, to an eye cover adapted for use with beauty treatments.

2. Description of the Related Art

Several designs for eye covers have been designed in the past. None of them, however, is known to include an easily adjustable device that holds, for example a cut vegetable, so that it is gently pressed up against the face in the eye region while retaining a high level of adjustability and adaptability for various different types and configurations of material to be held against the face.

Other devices require expensive materials. Some of these are difficult to adjust. Most other devices that may compete with the present design do not have readily available replacement parts so that if damaged or broken then the entire device is not useable and must be discarded and replaced with another device at significant additional cost to either the salon or the end user of the device.

Other patents describing the closest subject matter provide for a number of more or less complicated features that fail to solve the problem in an efficient and economical way. None of these patents suggest the novel features of the present invention.

### SUMMARY OF THE INVENTION

It is one of the main objects of the present invention to provide a device to simply hold a therapeutic slice of vegetative matter against both of one's eyes.

It is another object of this invention to provide a device that is sufficiently low cost as to make it essentially disposable. The low cost of the device allows a new unit to be used for each customer in a solon or other environment.

It is still another object of the present invention to provide a device to protect the wearer's eyes from damaging ultraviolet rays from the sun or a tanning booth while providing the cooling application of vegetable or fruit slices against the face.

It is yet another object of this invention to provide such a device that is inexpensive to manufacture and maintain while retaining its effectiveness.

Further objects of the invention will be brought out in the following part of the specification, wherein detailed description is for the purpose of fully disclosing the invention without placing limitations thereon.

### BRIEF DESCRIPTION OF THE DRAWINGS

With the above and other related objects in view, the invention consists in the details of construction and combination of parts as will be more fully understood from the following description, when read in conjunction with the accompanying drawings in which:

FIG. 1 represents a perspective view of a version of the device.

FIG. 2 shows a perspective view of a version of the device including a slice of cucumber.

FIG. 3 illustrates an example of how the device might be worn by a human.

FIG. 4 is a representation of a partial perspective view of a slice of fruit inside a version of an eyepiece.

FIG. 5 is a perspective view of an alternate version of the device including a slice of cucumber.

FIG. 6 is an elevation view of a version of an eyepiece that demonstrates an example of a graphic on an outside surface.

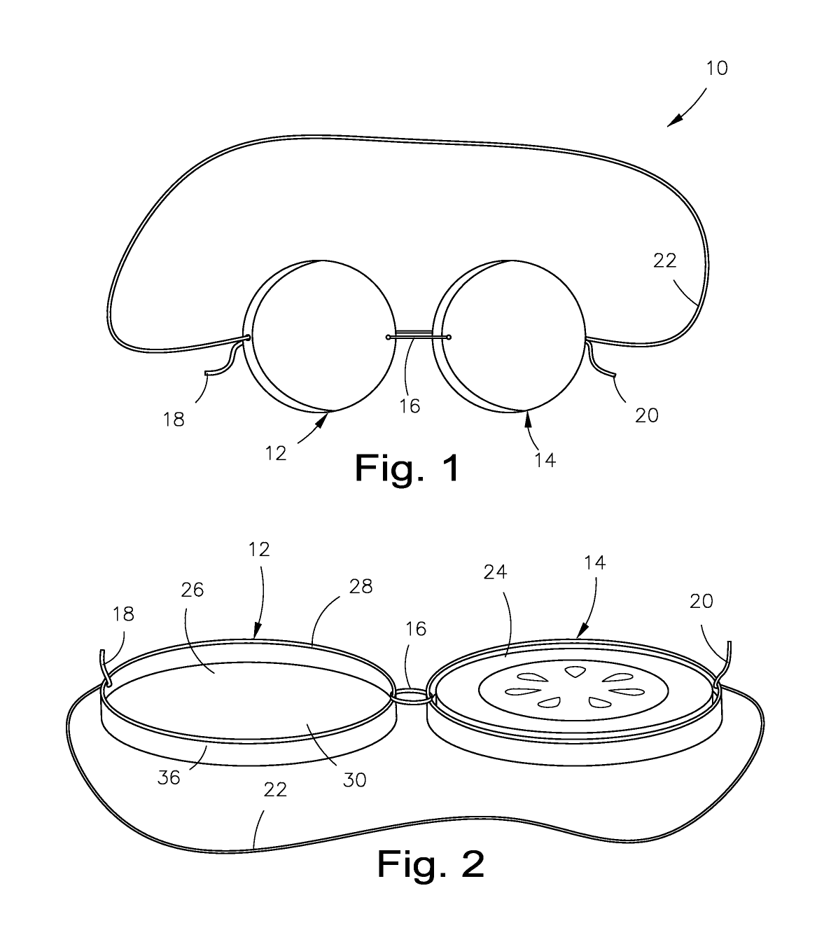

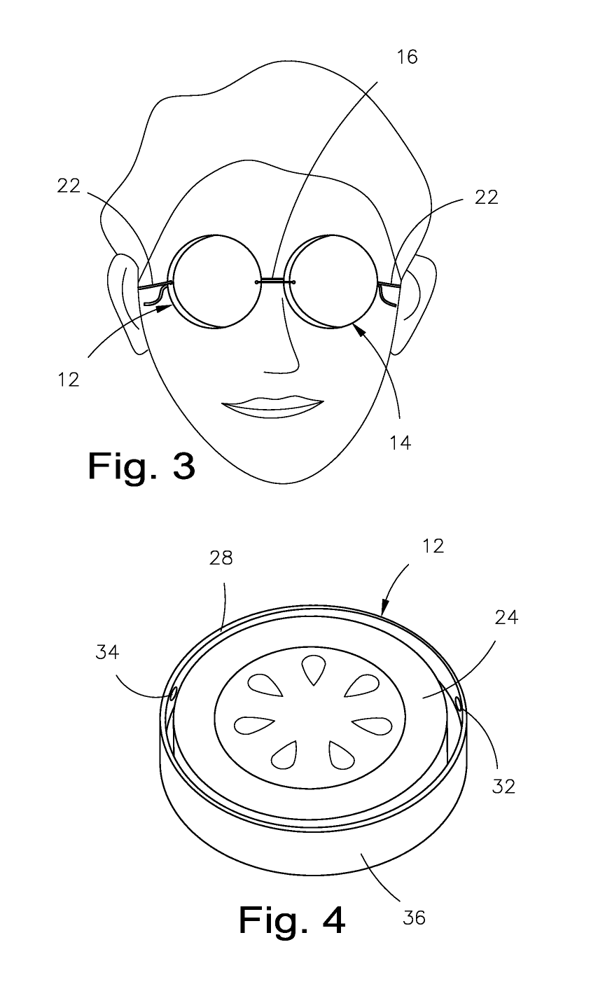

### DETAILED DESCRIPTION OF THE PREFERRED EMBODIMENT

In recent years it has become popular to relax and receive therapeutic treatments in salons and other similar service establishments. Skin treatments including tanning, rejuvenation and healing sessions are all common place.

Some modern procedures and techniques are best practiced by using cooling and protective articles over the eyes. Some methods employ mud, gel packs or, most commonly, slices of natural fruit or vegetables. Often, a combination of these objects, such as mud and vegetables, are used together for better effect.

Depending on the practitioner and desired results, a specific fruit or vegetable is called for in the prescribed therapy. For example, some believe that the essences comprising cucumber can tighten the skin and reduce wrinkles. Other remedies suggest citrus, potatoes, kiwi, papaya or tea bags be placed over the eyes.

During the course of these and related healing and care sessions the patient or recipient of the treatment is required to remain still and lie down to allow the practitioner access to the body and to keep the medicament applied to the eyes in place. The present invention relates to providing a treatment accessory to help keep the eye treatments held in place over the eyes which allows more freedom of movement and easier holding of the treatment on the eyes during sitting and other movement that the wearer might like to experience.

In particular, the present invention allows the wearer to keep both eyes simultaneously covered while holding the fruit of choice on the face. This permits the wearer to sit up in a chair which may be a much more comfortable position for many people. In other situations the patient can also be led by the practitioner to another part of the facility for continued treatment of a different variety without undue concern for the fruit pressed onto the patients' eyes.

In a preferred variation, the device is constructed from simple plastic and rubber elements so that the cost of the device is very low. A virtually disposable device is favorable to encourage use and maintain the sanitary nature of the device. Having it disposable also encourages its use and avoids the requirement of a cleaning regimen and cleaning equipment than if they are re-useable.

Referring now to the drawings, where the present invention is generally referred to with numeral 10, it can be observed that it basically includes in this iteration, as shown in figures one through four, an eyepiece 12, an eyepiece 14, a bridge 16, a tag 18, a tag 20, a headband 22, a fruit 24 (cucumber shown), a cover 26, a rim 28, a cavity 30, an aperture 32, an aperture 34 and an edge 36.

Figures five and six are presented to show a plurality of alternate and valuable features that may be included individually or in combination with any of the features shown in the other figures and described herein. The elements as demonstrated in figures five and six include, inter alia, an eyepiece 40, an eyepiece 42, a headband 44, a bridge 46, a fruit 48, an interior surface 50, a tag 52, a tag 54, an exterior surface 56, an exterior surface 58 and a graphic 60.

Referring to figure one, the eyepiece 12 is provided to cover one eye of a user of the device and the complementary eyepiece 14 is provided to cover the user's other eye. Eyepiece 12 is flexibly connected to eyepiece 14 by means of a bridge 16. The bridge 16 may be constructed of a thin rubber band, plastic wire, thread or other thin and flexible material. Care should be taken to select a material for the bridge 16 that is sufficiently strong to not break and remain flexible while being relatively economical and easy to manufacture.

A headband 22 also connects the eyepiece 12 and eyepiece 14. The headband 22 is preferably constructed of a stretchable material such as rubber or other elastic cord. The headband 22 is generally dimensioned sufficient to permit the device to encircle the head of almost any user of the device.

A shown in more detail in figure four, each eyepiece 12 and 14 has an aperture 32 and aperture 34 at roughly opposite sides of the respective eyepiece 12 and 14, near the edge of the respective eyepiece, that permits attachment of the headband 22 and bridge 16 to the eyepieces 12 and 14. The aperture 34 and aperture 36 may take the form of a single aperture each or may be double apertures that the bridge 16 and headband 22 can weave once in and then out to hold the bridge 16 and headband 22 in affixed position, thus a fixed length, relative to the eyepiece 12 and eyepiece 14.

Preferably the aperture 32 and aperture 34 are formed integral to the rim 28. However, in the version of the device as shown in figure five, where there is no feature analogous to the rim 28 as shown in figures one through four, there are apertures in the eyepiece 40 and eyepiece 42 respectively. And, similar to the aperture 32 and aperture 34, the apertures in the version shown in figures five and six may similarly be comprised of a single or multiple holes that can accept the headband 44 and bridge 46 to encircle the wearer's head.

Referring again to figures one through four, the cover 26 is preferably flexible so that it is comfortable when held over the eyes of the wearer. The cover is formed integrally to the rim 28 to define the cavity 30. The cavity is bounded by the edge 36 and the aperture 32 and aperture 34 are formed integral to the edge 36.

The cover 26 is dimensioned to be only thick enough to maintain its integrity. The cover 26 is dimensioned with sufficient width to both cover the eye socket of the wearer and to hold a piece of pre-selected fruit, vegetable or other therapeutic matter, such as those described above, in the cavity 30 formed by the rim 28 against the cover 26. In a valuable version of the device, the cover 26 is made of plastic. However, it may be possible to make the cover from other materials such as leather, wood, metal, fabric or any combination thereof.

Preferably the material from which the cover 26 and rim 28 is fabricated is somewhat flexible. Flexible sufficient so that the cover 26 may deform and the rim 28 may loosely come into contact about the eye orbit of the wearer. This is so that the fruit 24 is held up against the eyelids of the wearer and also, in case of use with a tanning bed, so that at least a partial light-proof seal with the skin is created to provide a limited degree of protection to the eyes.

In typical use the wearer of the device or treatment provider would select the appropriate material (i.e. fruit 24) to place inside the cavity 30. For this example we describe this material as a slice of cucumber but it could be any of a wide variety of available and homemade homeopathic articles that may comprise or complement the desired treatment. Some examples, but not an exhaustive list is provided supra.

Once the fruit 24 is in the cavity, the headband 22 is pulled over the back of the wearer's head while the fruit 24 is carefully kept over the wearer's eyes with the cover 26 holding the fruit directly over the wearers eyes. Once the device is loosely in place on the face then the tag 18 and tag 20 are each pulled to tighten the headband 22 around the head. Practically, either tag 18 or tag 20 may have the desired effect. However, pulling a little each on tag 18 and then tag 20 may avoid having the headband 22 pull on the wearer's hair.

Alternatively, the tag 18 and/or the tag 20 may be pulled to adjust the size of the headband 22 before attempting to put the device onto the head of the wearer. Typically the bridge 16 is not necessarily adjustable. The bridge 16 is normally factory preset at the distance needed to span the bridge of the wearer's nose.

In an important version of the device the exterior surface of the device (the opposite side of the eyepieces 12 and 14 that the cavity 30 is on) may be opaque and reflective. Having the exterior with reflective characteristics is especially beneficial when the device is used in combination with a tanning bed. The light from the tanner is thusly reflected and the fruit 24 is kept cooler. Additionally, the opacity of the reflective layer helps keep light out of the eyes of the wearer making for a more comfortable experience.

The diameter of the cover 26 is provided such that a fruit is available to conveniently cover the eye and so that the eye is otherwise covered with the cover 26. This frequently might be in the range of approximately one inch to about four inches. The precise dimensions can vary depending on the type of fruit needed for a particular treatment, the size of the wearer's face and eye orbits and the comfort needs of the patient or customer wearing the device.

Now referring to figures five and six where a variant of the device is demonstrated. The similarities of the several devices are readily apparent with the primary distinguishing features of this device including, among other things, the lack of a rim (such as the rim 28 in the earlier described versions). This permits the eyepiece 40 and eyepiece 42 to be more flexible than other versions.

This flexibility is even more so when the materials chosen for construction are selected to enhance this pliable characteristic. For example, plastic is a preferred material for the eyepiece 40 and 42. However, other materials such as leather, metal, fabric or any combination of these may also be chosen depending on the ultimate use of the device and if it is to be disposable or re-useable.

Another advantage of the rim-less version of the device is that a wider variety of fruit may be used. For example, fruit may come in different sizes that the rim-less design may accommodate easier. Further, less material and simpler construction techniques permitted by not having a rim reduces the production costs and makes a disposable version even more feasible.

The bridge 46, headband 44, tag 52 and tag 54 function materially similar to the similarly identified elements in other versions described supra. Similarly, the fruit 48 may be any of the earlier identified fruits or other treatment modes. The use and variations of this variety are also materially similar.

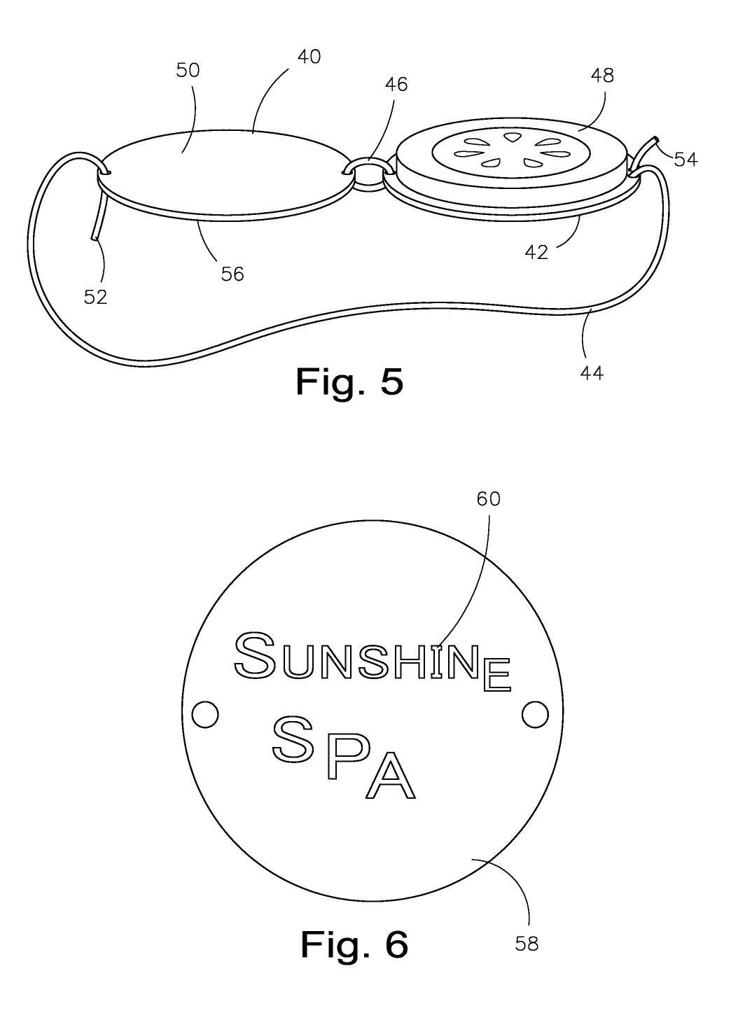

The eyepiece that covers the left eye is identical to the one that covers the right eye. The principal difference being that for one the bridge 46 attaches to left side and the headband to the other where the other eyepiece connects to the other elements vice-versa. This is true for both the rimmed version in figure one and the rim-less version in FIG. 5. When constructed no difference between the left and right side need be made until the components are assembled.

Figure six shows the exterior surface 58 of a single eyepiece. This is in contrast to the interior surface 50 (shown in figure five). Exterior surface 56 and exterior surface 58 are analogous elements shown between the views in figures five and six.

The exterior surface 58 has applied to it an optional graphic 60. This graphic 60 may be applied over the surface, embossed or any other commonly available means to add artistic or verbal embellishment to the exterior surface 58. Typically, the graphic might be an advertisement for the salon or spa providing the device to its customer or it may indicate the brand of the device provider. The ambidextrous nature of the eyepieces also permit the graphic 60 to be flipped to the interior, against the fruit, if desired by the user.

The invention may also be fairly described as an eye cover comprised of a first eyepiece, a second eyepiece, a headband and a bridge. The first eyepiece and said second eyepiece each have a first aperture at a first position dimensioned to connect to said bridge and a second aperture at a second position dimensioned to connect to said headband. Basically, the apertures are each a hole or pair of holes that the bridge threads into. The bridge is a short cord or rubber band that essentially connects the two eyepieces over the bridge of the nose. The first eyepiece at said first aperture and said second eyepiece at said first aperture are flexibly connected to each other by the bridge. The length of the bridge may be fixed or flexible and may be comprised of one length of material or a loop of material. The first eyepiece at said second aperture and said second eyepiece at said second aperture are connected to each other by the headband. Basically, these apertures are either a single or double hole in the eyepieces that connect the outside edge of each eyepiece with an elastic rubber band or other similar stretchy material. If the design includes two holes for each of these apertures then the headband can be looped through both holes and pulled tight to custom fit the headband to that user. The first eyepiece and said second eyepiece each have an interior surface and an exterior surface. Typically a slice of fruit is places on the interior surface where it is pressed up against the user's eye. The juice in the fruit can then interact with the skin. The interior surfaces and exterior surface are coplanar and are dimensioned similar to a preselected slice of fruit. Generally, the eyepieces are made from a sheet of plastic material and the several apertures are stamped out of them.

In another similar version of the device a rim is affixed to said interior surface around a periphery of each of said first eyepiece and said second eyepiece. This rim serves to aid in holding the slice of fruit against the eye. Since some fruit is a little slippery the rim can keep the fruit from slipping out from between the eye and the eyepiece. In another variation of the device the exterior surface of either of said first eyepiece and/or said second eyepiece includes a graphic. This graphic might be a trademark or advertisement. Contemplated uses might be to advertise the salon or salon products. In this way the cost of the product might be subsidized by the advertising revenue. In another version of the device the length of said headband is adjustable. For example, a pair of apertures can be used to weave the end of the headband into leaving a tag end of the headband. This tag end may then be pulled, drawing a portion of the length of the headband through the apertures and thereby shortening the length of the headband to fit various sized heads. Another variation of the device is that where the headband is constructed of rubber or synthetic rubber (i.e. a rubber band-type material) and both of said eyepieces are made of plastic. Plastic may be preferred because of its flexibility, wash-ability, sanitary nature and low cost.

The foregoing description conveys the best understanding of the objectives and advantages of the present invention. Different embodiments may be made of the inventive concept of this invention. It is to be understood that all matter disclosed herein is to be interpreted merely as illustrative, and not in a limiting sense.

What is claimed is:

1. An eye cover comprised of a first eyepiece, a second eyepiece, a headband and a bridge;
Said first eyepiece and said second eyepiece each has a first aperture at a first position dimensioned to connect to said bridge and a second aperture at a second position dimensioned to connect to said headband;
said first eyepiece at said first aperture and said second eyepiece at said first aperture are flexibly connected to each other by the bridge;
said first eyepiece at said second aperture and said second eyepiece at said second aperture are connected to each other by the headband;
said first eyepiece and said second eyepiece each have an interior surface and an exterior surface;
said interior surfaces and exterior surface are coplanar and are dimensioned similar to a preselected slice of fruit.

2. An eye cover as described in claim one further characterized in that a rim is affixed to said interior surface around a periphery of each of said first eyepiece and said second eyepiece.

3. An eye cover as described in claim one further characterized in that said exterior surface of either of said first eyepiece and/or said second eyepiece includes a graphic.

4. An eye cover as described in claim one further characterized in that the length of said headband is adjustable.

5. An eye cover as described in claim one further characterized in that said headband is constructed of rubber or synthetic rubber and both of said eyepieces are made of plastic.
